(19) United States
(12) Patent Application Publication
(10) Pub. No.: US 2013/0338712 A1
(43) Pub. Date: Dec. 19, 2013

(54) DEVICE FOR CORRECTING SCOLIOSIS AND CONTROLLING VERTEBRAL ARTHRODESIS

(71) Applicants: Michel Massenzio, Lentilly (FR); Sylvie Ronel, Villeurbanne (FR); Vincent Cunin, Begnoit (FR); Abdelkrim Bennani, Villeurbanne (FR); Eric Jacquelin, Miribel (FR); Yoann Lafon, Villeurbanne (FR)

(72) Inventors: Michel Massenzio, Lentilly (FR); Sylvie Ronel, Villeurbanne (FR); Vincent Cunin, Begnoit (FR); Abdelkrim Bennani, Villeurbanne (FR); Eric Jacquelin, Miribel (FR); Yoann Lafon, Villeurbanne (FR)

(73) Assignee: UNIVERSITE CLAUDE BERNARD LYON I, VILLEURBANE CEDEX (FR)

(21) Appl. No.: 13/997,283

(22) PCT Filed: Dec. 16, 2011
(86) PCT No.: PCT/FR2011/053019

§ 371 Date: Aug. 28, 2013

(30) Foreign Application Priority Data
Dec. 23, 2010 (FR) 1061205

(51) Int. Cl. A61B 17/70

(52) U.S. Cl. CPC A61B 17/7052; A61B 17/7047
USPC 606/252; 606/251

(57) ABSTRACT

The present invention provides a scoliosis correction device comprising a pair of distraction rods (2, 3) and a member (4) for linking the distraction rods provided with means (8) for guiding each of the rods relative to each other inside said linking member. This device further comprises at least one member (6) for suspension of the distraction rods (2, 3) that can provide flexibility and exert a longitudinal pre-load on the rods in order to augment comfort and prevent vertebral arthrodesis of the spine of a patient instrumented with the correction device. Finally, it comprises a structure for cushioning and locking the distraction rods in translation in the linking member, said structure being active only when compressive forces are applied by the spine of a patient to the distraction rods (2, 3) so as to absorb and oppose said compressive forces and augment patient comfort, thereby preventing vertebral arthrodesis of the patient's spine.

### FIELD OF THE INVENTION

The present invention relates to the field of correcting scoliosis, in particular infantile scoliotic pathologies.

Scoliosis is a three-dimensional deformation of the vertebral column that usually occurs during growth. Severe scoliosis may result in substantial deformation with arthrosis, cardio-respiratory repercussions and esthetic unacceptability.

Scoliosis progression is maximal during growth. It tends to become stabilized in the adult. The majority of treatments are thus directed towards children or adolescents. Severe forms require corseting treatment and sometimes surgical intervention.

### PRIOR ART

Scoliosis surgery is known as vertebral arthrodesis. It is intended to lock the vertebrae together, stop progress and straighten the vertebral column as much as possible with the aid of metal rods attached to the column. The principal disadvantage of such surgery is that it causes stiffening of the vertebral column and stops growth in the operated zone. Thus, arthrodesis procedures have to be carried out when a patient has finished growing. In addition, the surgery is not corrective but is palliative, fastening the column in the best possible position for the patient.

Certain infantile and juvenile scolioses progress “malignantly” because they resist corseting orthopedic treatment and progress inexorably to major deformations in adulthood, with orthopedic consequences that have a huge effect on the patient's vital functions. Such scolioses cannot be treated by conventional arthrodesis, which blocks the scoliosis but also blocks growth. At the same time, orthopedic treatments carried out on such scolioses provide mediocre results.

In addition, in order to treat such infantile scoliotic pathologies, various corrective surgical techniques have been developed and are in current use.

A first technique that is widely used in scoliosis correction treatments is the growth rod or distraction rod technique. In that technique, the ends of one or two distraction rods are attached to one vertebra of the portion above the deformed zone of the spine and to one vertebra of the portion below the deformed zone of the spine. The length of the implanted rod or rods is slightly greater than that of the imaginary line linking the two attachment points of the rod to the spine, such that the rod has the effect of pushing the adjacent portions of the spine back, forcing the spine to straighten.

However, a major disadvantage of that technique arises when the rods have to be lengthened as the implanted patients grow, and thus regular surgical procedures have to be carried out on the patients, approximately every 4 to 6 months, in order to change the rods or at the very least to lengthen them. Those repeat interventions are difficult for the patient and family to accept; in addition, the force that has to be applied during each intervention in order to make the rod longer is proportional to the stiffening of the vertebral column, which loses its flexibility due to the scoliosis and also due to the immobility caused by the rod.

In addition, implants known as VEPTR™ (vertical expandable prosthetic titanium rib) are known. Such implants are very similar in their form and function to conventional distraction rods. In addition, they are more robust. However, they also suffer from the same disadvantages as distraction rods, in particular the necessity for regular repeat surgical intervention in order to lengthen the implant to match the patient's growth.

That operational constraint, linked to the need to lengthen distraction rods and implants, has been the focus of several attempts at solutions; unfortunately, until now, none of them appears to have been shown to be effective.

In particular, a Japanese team proposed a growth rod device that enlarges under the control of a motor (M. Takaso et al: Growing-rod spinal instrumentation, Journal of Orthopedic Sciences (1998), 3:336-340).

More recently, a Spanish research team has also proposed a transcutaneously activated electromechanical correction implant (A. M. Pernia et al, Mechatronics 18 (2008), 616-626).

A system (Phenix® rod) is also known that makes use of a rod that enlarges under the action of a magnet that is regularly passed over the back of the patient. That system, which in theory appears to be effective, is however very expensive, necessarily preventing any application thereof with a majority of patients.

Finally, a controlled growth correction system is described in document US 2009/0204156 A1, which comprises a distraction rod correction device linked together via a system for releasably engaging the rods, said system allowing the rods that are fastened to the spine to slide during growth stages and allowing those rods to lock outside those stages, in particular by an over-center reaction. However, that correction system has proved to be complex to produce and, despite its capacity for rod extension, it is extremely rigid; that may prove to be a problem for the patients.

### DISCLOSURE OF THE INVENTION

Firstly, the aim of the invention is to provide a device for correcting scoliosis that can be used to prevent vertebral arthrodesis in a simple manner.

Secondly, the aim of the invention is to provide a device for correcting scoliosis, in particular for a child, which can automatically be adapted to growth of the child without requiring an invasive or non-invasive medical intervention for lengthening the correction device to match the patient's growth, after placing it on the patient.

A further aim of the invention is to provide a scoliosis correction device that can be used to produce more gradual, and therefore more effective, distraction of the vertebral column.

In particular, the aim of the invention is to provide a device that can be used to correct a scoliosis without arthrodesis at the end of growth, meaning thereby that the physiological role of mobility of the vertebral column is maintained.

The invention also aims to provide a correction device that is simple in structure and thus has a reasonable manufacturing and fitting cost for the patients.

In accordance with the invention, these aims are achieved by means of a device for correcting scoliosis comprising at least:
a pair of distraction rods; and
a distraction rod linking member provided with means for guiding each of the rods relative to each other inside said linking member.

This device is characterized in that it comprises, for each distraction rod:
at least one suspension member for the distraction rods that is capable of providing flexibility and exerting a longitudinal pre-load on the distraction rods; and
a structure for cushioning and locking the distraction rods in translation in the linking member, said structure being active only when compressive forces are applied by the spine of a patient to the distraction rods so as to absorb and oppose said compressive forces and augment the comfort of the patient, thereby preventing arthrodesis of the vertebrae of the patient's spine.

The correction device of the invention has the major advantage of allowing the scoliosis of the spine of an instrumented patient to be corrected dynamically and gradually.

In fact, the suspension member and the structure for cushioning and locking the rods can endow the correction device with a capacity to support and correct the spine and at the same time can provide for longitudinal flexibility between the distraction rods. This “flexible locking” capacity of the distraction rods can be used to control vertebral arthrodesis by allowing a small amount of play between the plates and vertebral disks in the zone of the instrumented spine, which reflects anatomical reality. Thus, the correction device has rigidity appropriate to countering the deformation process of the spine linked to the scoliosis in a manner similar to that produced by conventional distraction rods. At the same time, the device can overcome the disadvantages linked to the rigidity of the corrective system by means of the suspension that is produced.

In accordance with a first feature of the invention, the linking member comprises a casing inside which at least one said suspension member for each rod is housed and the rods are inserted into said casing and each cooperates with a said suspension member.

Such a structure proves to be particularly compact and simple to produce, which facilitates implantation and limits production costs.

In accordance with another feature of the device of the invention, the distraction rods are introduced into and extend along two parallel axes in the linking member.

In an advantageous embodiment, the structure for cushioning and locking the correction device comprises, for each distraction rod, a ring for adjusting the positions of the rods relative to the linking member, each said ring being mounted coaxially and, being slidably movable on each said rod, and cooperating with at least one means for locking against sliding on the rods in at least one direction.

In this embodiment, each adjusting ring bears on at least one said suspension member mounted between an internal wall of the linking member and said adjusting ring.

In accordance with a first variation of this embodiment, the locking means comprise a screw inserted into an aperture formed in the adjusting ring.

In accordance with another variation of this embodiment, the locking means comprise a toothed washer.

In accordance with a third variation of this embodiment, the locking means consist of an assembly comprising a split ring and a disk clamp slidably mounted on the distraction rod inside an at least partially conical chamber formed in the adjusting ring, such that the disk clamp presses on the split ring in a conical section of the chamber.

In this third variation, the disk clamp cooperates with a return means housed in a non-conical section of the chamber formed in the adjusting ring.

In a fourth variation of this particular embodiment of the invention, the locking means consist of an assembly comprising rollers and a disk clamp slidably mounted on the distraction rod inside an at least partially conical chamber formed in the adjusting ring, such that the disk clamp presses on the rollers in a conical section of the chamber.

Advantageously, in this variant embodiment, the disk clamp cooperates with a return means positioned between the disk clamp and an internal wall of the chamber perpendicular to the axis of the rod.

Advantageously again, the return means of the disk clamp comprise a helical spring mounted coaxially with the distraction rod in said chamber of each ring.

In an alternative embodiment of the invention, the structure for cushioning and locking the distraction rods comprises an over-center device for locking the distraction rods relative to the linking member.

In accordance with this embodiment of the device of the invention, the device for locking the distraction device advantageously comprises two keepers that are movable in translation in the linking member in a direction parallel to the axes Xl, X2 in the linking member, said keepers being disposed either side of the suspension member and each bearing both on said suspension member and also on a distraction rod in order to lock it against sliding in a first direction relative to the linking member by an over-center effect while being capable of allowing it to slide freely in a second direction opposite to the first during stages of growth of a spine instrumented with the correction device.

Advantageously again in this variant embodiment, each keeper of the locking device comprises a cam pivoting on a support body bearing on the suspension member, the cam cooperating with a resilient return element that is attached to the support body and that constrains said cam to bear on its cam surface against a distraction rod.

Yet more advantageously, the keepers and the suspension member are held and are movable parallel to the distraction rods in a guide profile provided in the linking member.

Finally, as is conventional and, of course, essential with implantable medical devices, the linking member and the distraction rods are made out of biocompatible material, preferably a metal.

### BRIEF DESCRIPTION OF THE DRAWINGS

Various other characteristics become apparent from the following description made with reference to the accompanying drawings that show embodiments of the subject matter of the invention, by way of non-limiting examples.

FIGS. 1A to 1C show an embodiment of a correction device in accordance with the invention;

FIGS. 2A to 2C show a variation of the embodiment of the correction device shown in FIGS. 1A to 1C;

FIGS. 3A and 3B show a variant embodiment of the correction device shown in FIGS. 1A to 1C;

FIGS. 4A and 4B show another variant embodiment of the correction device shown in FIGS. 1A to 1C;

FIGS. 5A to 5C show a second embodiment of a correction device in accordance with the invention.

### DESCRIPTION OF EMBODIMENTS OF THE INVENTION

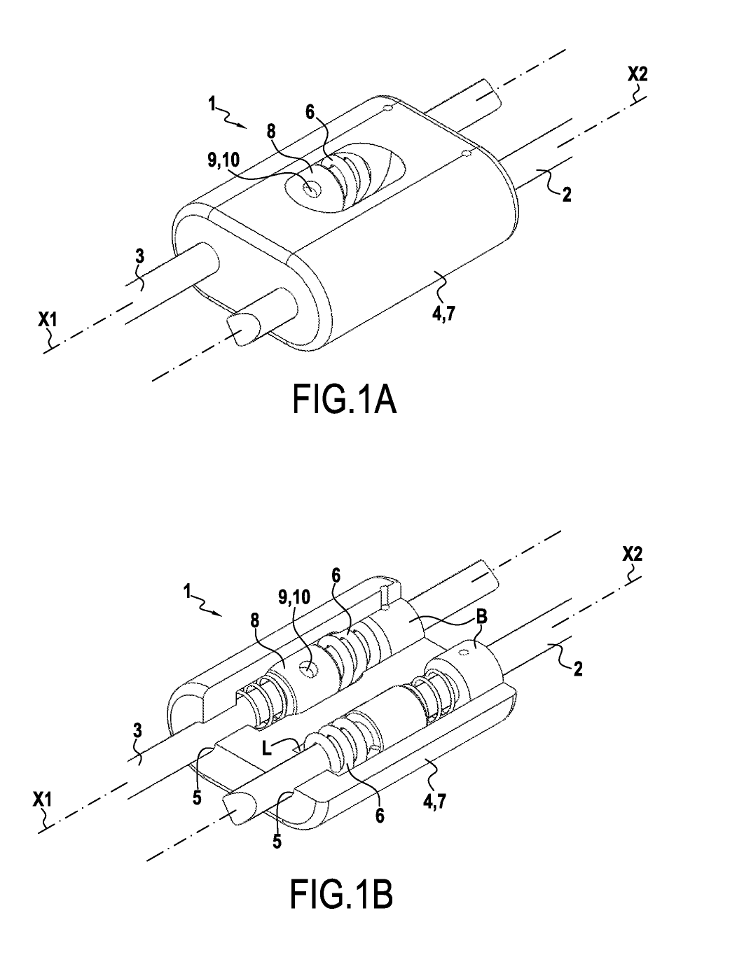

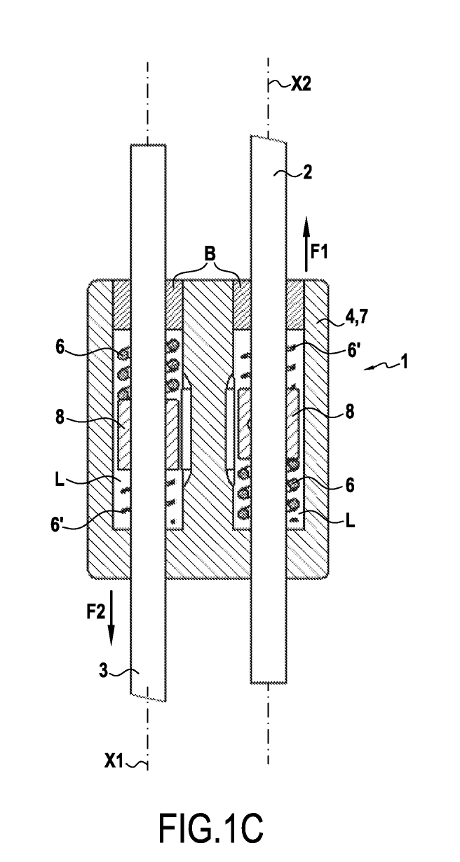

FIGS. 1A to 5C show, in detail, various embodiments and variant embodiments of a device 1 for correcting scoliosis in accordance with the present invention, in increasing order of complexity and function.

Irrespective of the embodiment or the variant embodiment under consideration, the scoliosis correction device 1 shown essentially comprises a pair of distraction rods 2, 3, and a linking member 4 for said distraction rods 2, 3, provided with guide means comprising at least respective channels 5 for inserting and guiding each of the rods 2, 3 relative to each other inside the linking member 4.

The two distraction rods 2, 3 are normal distraction rods. They are each provided with means for anchoring to vertebrae, such as screws and hooks, which are also normally used for fastening such distraction rods to a spine, a first rod being fastened via an upper end to the vertebra above the deformed portion of a spine to be corrected, the other rod being fastened via a lower end to the vertebra below the deformed portion.

The linking member 4 is also advantageously constituted by a casing 7 cut with orifices and channels 5 for passing distraction rods 2, 3 in the casing 7, said rods 2, 3 being able to slide freely in these orifices and channels. The linking member 4 of the distraction rods may be formed by a casing with a non-rectangular shape, for example egg-shaped or any other non-geometrical shape, the particular shape of the coupling member not being of particular functional importance apart from being able to have the distraction rods 2, 3 inserted in a slidable manner and to provide therein an internal chamber for receiving at least one rod suspension member as is described below.

The correction device 1 also comprises at least one suspension member 6 for the distraction rods 2, 3, which member can provide flexibility and exert a longitudinal pre-load on the rods 2, 3 in order to augment comfort and prevent vertebral arthrodesis of the spine of a patient instrumented by means of a correction device 1.

The suspension member 6 is advantageously formed by a spring, which may be helical or of another type, for example, that is attached to or at the very least in contact with the distraction rods 2, 3 and that exerts a minimum reaction force thereon tending to push on the rods in two opposing directions F1, F2 to promote distraction of the spine and correction of the scoliotic portion of the instrumented spine.

At the same time, said suspension member 6, which is resilient, may be constrained by the rods during movement of the spine, which means that the correction device 1 is provided with intrinsic flexibility, which is therefore close to anatomical reality where the inter-vertebral disks provide the entirety of the vertebral column with flexibility.

Finally, the correction device 1 comprises a structure for cushioning and locking the distraction rods 2, 3 in translation in the linking member 4. This cushioning and locking structure may be produced in a variety of forms, as is described below in FIGS. 1A to 5C. It is advantageously designed to be active in locking the rods only when compressive forces are exerted by the spine of a patient on the distraction rods 2, 3 so that they absorb and oppose the compressive forces. When tensile forces are exerted by the spine on the rods 2, 3, for example during growth of the spine, the cushioning structure is “free”, i.e. it allows the rods 2, 3 to slide in the linking member, allowing free growth. The correction device 1 of the invention can thus be used to augment the patient's comfort, thereby avoiding vertebral arthrodesis of the patient's spine.

In a first embodiment shown in FIGS. 1A to 10, the correction device 1 comprises a casing 7 in which each of the two distraction rods 2, 3 of the correction device 1 is inserted along its own axis X1, X2, these two axes X1, X2 being parallel.

The casing 7 has a block-like, substantially parallelepipedal shape, in which two insertion and guiding channels 5 are formed for the distraction rods 2, 3 to slide therein. These two channels 5 are parallel and they both open out in the longitudinal direction of the casing 7, i.e. parallel to the distraction rods 2, 3.

Inside the casing 7, two cylindrical housings L are formed that are coaxial with the channels 5 for guiding the distraction rods 2, 3. Each housing L is for receiving and accommodating a suspension element 6 of the corresponding distraction rod 2, 3 and a structure for cushioning and locking the rod, here comprising a ring 8 for adjusting the travel of the distraction rod 2, 3 in the casing 7.

The housings L in the casing 7 are accessible by removing a closing plate of the casing 7 concealing access to the inside of said casing 7. This closing plate is advantageously screwed onto the casing 7 and onto plugs B for sealing the housings L at one longitudinal end of the casing 7. These plugs B are threaded onto each of the distraction rods 2, 3, as can be seen in FIGS. 1B and 1C in particular, and they plug the housings L of the casing 7 to prevent the adjusting ring 8 and the suspension element 6 in each housing L from coming out.

As can be seen in FIGS. 1B and 1C, each distraction rod 2, 3 cooperates in a housing L of the casing 7 with a cushioning and locking structure comprising a ring 8 for adjusting the travel of the longitudinal movement of that rod in its housing L. This adjusting ring 8 is threaded onto its distraction rod 2, 3 and held in a predetermined position by means of a locking means 9 such as a screw 10, for example.

Furthermore, either side of the adjusting ring 8, each distraction rod cooperates in its housing L with a suspension element 6 formed in the example shown as a helical spring, bearing on one end of the housing L and wound about the distraction rod 2, 3, and a compression spring 6′, which is complementary to the suspension element 6 and less stiff, wound about the distraction rod 2, 3 on the other side of the adjusting ring 8, which is thus flanked by the springs 6, 6′ in its housing L.

Advantageously and for the correction device 1 to be properly effective, the position of the suspension spring 6 in a first housing L is reversed relative to the position of the suspension spring 6 in the other housing L of the casing, and likewise for the complementary compression springs 6′. This means that the suspension of each of the distraction rods 2, 3 is effective and optimal in order to accommodate longitudinal compressive loads from the spine in the instrumented zone.

The springs 6′ are not essential. They provide for a transition of contact by the reserve length of rods 2, 3 when growth has finished.

In contrast, the suspension springs 6 per se are indeed essential. They exert a pre-load on the distraction rods 2, 3 promoting scoliosis correction. To this end, when fitting the correction device to the patient, they are advantageously compressed a little with the aid of the adjusting ring 8, which can be moved on the distraction rods 2, 3 for this purpose; this also adjusts the length of the movement travel of the rods 2, 3 as the instrumented patient grows, if the patient is still growing.

In fact, because of the adjusting rings 8, the correction device 1 can be used to provide the distraction rods 2, 3 with a capacity for elongation within their respective housings L in the casing 7 in order to follow the growth of the instrumented patient.

In similar manner, in order to pre-load the suspension elements 6, this adjustment can in particular be made through a central port in the closing plate of the casing 7.

Once the travel and the pre-loading on the suspension springs 6 have been adjusted, the distraction rods 2, 3 can be used both to elongate with growth of the patient in the direction of the arrows F1, F2 in FIG. 1C, and to accommodate any compressive forces on the spine in the direction opposite to the arrows F1, F2 due to the combination of the adjusting rings 8 and suspension elements 6 in each of the housings L in the casing 7.

The scoliosis correction device 1 of the invention in this variant embodiment thus provides an in situ, adjustable extensible correction solution; in addition, because of its extremely simple structure, fitting, function and any maintenance of the device proves to be extremely simple and reliable.

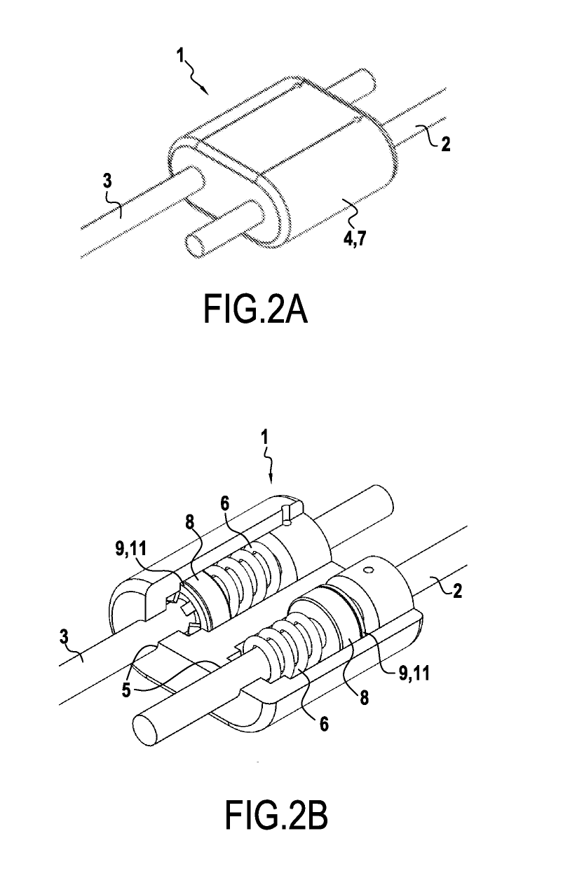

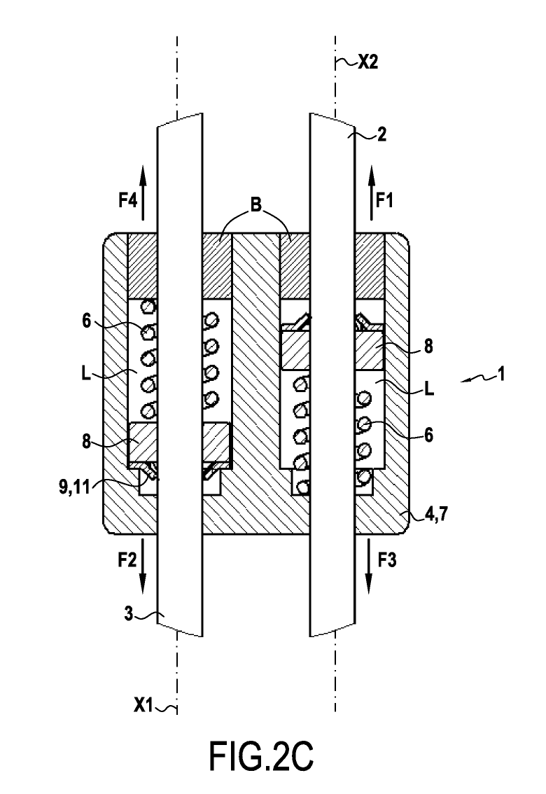

A second variant embodiment of the correction device 1 of the invention is presented in FIGS. 2A to 2C. This variant embodiment is analogous in its function and form to the preceding variation of FIGS. 1A to 1C. However, it differs in that it no longer includes additional compression springs 6′ and in that the adjusting rings 8 are now freely slidably mounted on the distraction rods 2, 3 in the housings L of the casing 7, locking of said adjusting rings 8 on the rods being provided for by a locking member 9 that this time is formed not by a screw, but by a toothed washer 11 attached directly to the adjusting ring 8.

Advantageously in this variant embodiment, the use of a toothed washer 11 in the guise of a means 9 for locking the adjusting rings 8 dispenses with the need for access to the interior of the casing in order to adjust the position of the rings 8 and the pre-load on the suspension elements 6. In fact, said adjustment is made only during implantation of the device 1 into a patient then no longer needs to be modified; the teeth of the toothed washer 11 are advantageously oriented in the growth direction of the rods 2, 3 on which they are mounted so that, during stages of spine growth, they allow the rods to slide in the rings 8, the washers 11, and the casing 7, in the direction of the arrows F1, F2 as shown in FIG. 2C.

At the same time, outside such spinal growth stages, the teeth of the toothed washers 11 rub on the rods 2, 3 and lock them against sliding in the direction F3, F4 opposite to the arrows F1, F2 by an over-center effect. This locking then gives rise to a reactional bearing force on the adjusting rings 8 that bear on the suspension elements 6 in order to flexibly support the longitudinal compressive load on the distraction rods 2, 3 and carry out the function of distraction of the rods, while preventing locking that is the origin of vertebral arthrodesis.

Thus, in this variant embodiment of FIGS. 2A to 2C, a scoliosis correction device 1 is obtained that is self-adapting to the growth of instrumented patients.

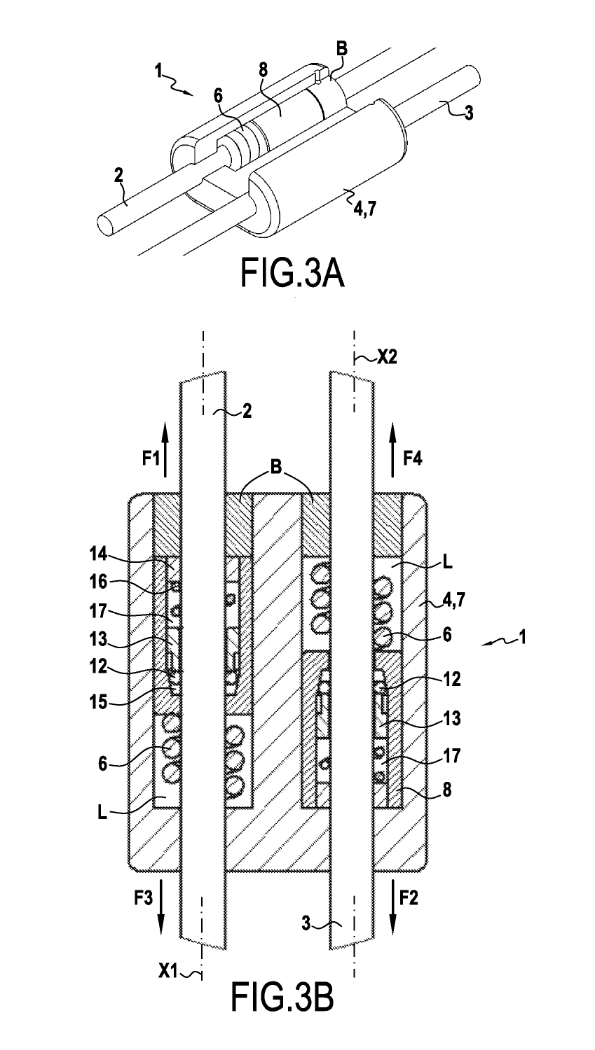

Two other variant embodiments of self-adapting scoliosis correction devices 1 in accordance with the invention are described below and shown in FIGS. 3A to 3C then 4A and 4B.

Firstly, FIGS. 3A to 3C show a self-adapting scoliosis correction device 1 analogous to that of FIGS. 2A to 2C but with a more integrated structure, in particular as regards the cushioning and locking structure of the distraction rods 2, 3. In fact, the cushioning and locking structure formed for each distraction rod 2, 3 by an adjusting ring 8 and a locking toothed washer 11 in the variant embodiment of FIGS. 2A to 2C is replaced in the variation of FIGS. 3A and 3B by a cushioning and locking structure formed by a tubular adjusting ring 8 threaded onto each distraction rod 2, 3 and placed in a housing L inside the casing 7, said ring defining in its body a housing comprising a cylindrical portion 17 and a conical portion 15 housing a spring 16, a disk clamp 13 and a split ring 12 positioned in the conical section 15.

The ring 8 in each housing L of the casing is also in contact with a suspension element 6 formed by a helical spring as shown, or a Belleville spring washer or an elastomeric material, for example. This suspension element 6 bears on an internal surface of the casing 7.

As can be seen in FIG. 3B, the two adjusting rings 8 are threaded onto each rod 2, 3 such that the diameter of their internal conical section decreases in a direction opposite to the direction F1 and F2 of movement of the distraction rods 2, 3 during growth stages of the spine of the patient instrumented with the correction device 1 of the invention.

Thus, using this construction arrangement, the distraction rods 2, 3 can slide in the direction of the arrows F1, F2 during spine growth stages and, in contrast, this sliding is locked in the direction of the arrows F3, F4 by a wedging effect of the split rings 12 pressed by the disk clamp 13 under the effect of the springs 16 in the conical sections 15 of the internal housing of the adjusting rings 8.

The springs 16 are judiciously pre-loaded on the disk clamps 13 inside the adjusting rings by a loading plate 14 closing the internal housing of the adjusting rings bearing on the springs 16 which themselves bear on the disk clamps and the split rings 12.

Thus, the default situation is that the assembly tends to be locked on the distraction rods 2, 3, and only under the effect of traction on the rods 2, 3 during growth stages of the spine will the friction of the rods 2, 3 on the split rings 12 inside the conical sections 15 of the rings 8 tend to cause said split rings 12 and disk clamps 13 to lift, causing the wedging effect to be released and allowing the distraction rods 2, 3 to lengthen.

As soon as growth stops, the stiffness of the springs 16 once again becomes greater than the friction of the rods 2, 3 on the split rings 12, which are again urged back by the disk clamps 13 at the bottom of the conical section 15 of the adjusting rings 8, thereby locking said adjusting rings 8 on the distraction rods 2, 3. With a compressive load on the rods 2, 3 by the spine tending to urge the rods back in the direction of the arrows F3, F4, the adjusting rings 8 then bear on the suspension springs 6 to render the correction device 1 flexible and accommodate the force applied for better patient comfort and integrity of the correction device 1.

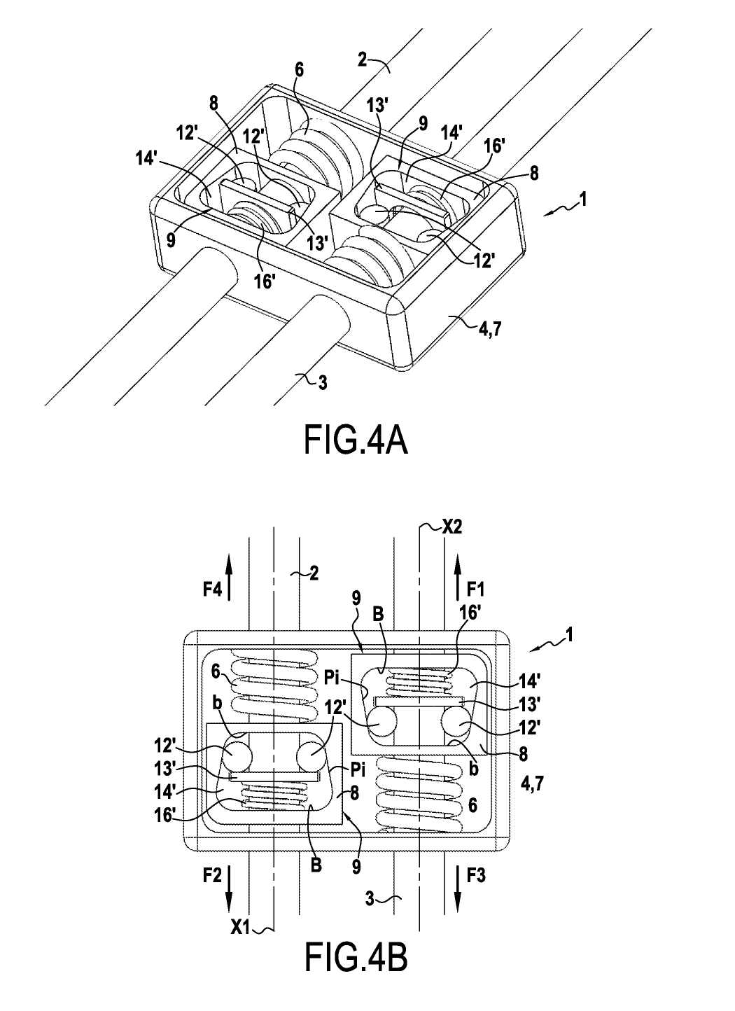

A fourth embodiment of the correction device 1 of the invention, which is structurally analogous to the three embodiments described above, is presented in FIGS. 4A and 4B.

In this embodiment, the scoliosis correction device 1 comprises a linking member 4 formed by a casing 7 with a rectangular section in a plane containing the axes X1, X2 extending the distraction rods 2, 3 each inserted into apertures or channels formed along these parallel axes X1, X2 in two opposite walls of the of the casing 7. A rectangular housing is formed inside the casing 7. In this housing, the distraction rods 2, 3 extend along the axes X1, X2. Each of the rods 2, 3 cooperates with a cushioning and locking structure, here comprising a ring 8 and a suspension spring 6 threaded onto each distraction rod 2, 3. Inside the housing, the ring 8 and the spring 6 carried by the rod 2 are threaded onto it in a position that is reversed relative to the ring 8 and the spring 6 on the rod 3.

In this particular embodiment, each adjusting ring 8 is more particularly formed as a block comprising, in a traverse plane containing the axes X1, X2 extending the rods 2, 3, an internal partially conical chamber 14′, more exactly with an isosceles trapezoidal section. The small base b of this isosceles trapezoidal section is oriented in an opposing direction for each of said rings 8. In the figure, the base b of the ring 8 on the rod 2 is oriented in the direction of the arrow F4, while the base b of the ring 8 on the rod 3 is oriented in the direction of the arrow F3.

Advantageously, the means 9 for locking the rods 2, 3 in this embodiment consist of an assembly comprising a disk clamp 13′ that is slidably mounted on each distraction rod 2, 3 inside the chamber 14′ in each adjusting ring 8. On a first face opposite to the small base b of the isosceles trapezoidal section of the chamber 14′, the disk clamp 13′ bears on rollers 12′ each disposed between an inclined plane Pi and the distraction rod 2, 3 and on a second face opposite to the large base B of the isosceles trapezoidal section of the chamber 14′, on at least one return means, for example a helical spring 16′, positioned between said disk clamp 13′ and the large base B of the trapezoidal section such that the disk clamp 13′ urges the rollers 12′ towards the small base b of the trapezoidal section of the chamber.

As can be seen in FIG. 4B, each ring 8 advantageously bears on the suspension spring 6 on the side of the small base b of the trapezoidal section of the chamber 14′. Clearly, care should be taken to select the suspension springs 6 and return springs 16′ such that the stiffness constant k of the suspension spring 6 is significantly larger than that of the return spring 16′ of the disk clamp 13′.

Thus, when a tensile stress is applied to the rods 2, 3 in the direction of the arrows F4, F3 respectively, in particular during growth stages of the spine of a patient equipped with the device 1, each rod 2, 3 is initially locked by the rollers 12′ in the chambers 14′ of the rings 8. The rings 8 then compress the suspension springs 6, which are of stiffness that is greater than that of the return spring 16′ of the disk clamp 13′. The return spring 16′ is then in turn compressed, allowing the rollers 12′ to be released and allowing the distraction rod 2, 3 to slide. The correction device 1 can thus allow dynamic correction of a spinal deformation.

In contrast, when a compressive force is applied to the distraction rods 2, 3 in the direction of the arrows F2 and F1 respectively, in particular outside the growth stages of the equipped patient's spine, the suspension springs 6 press on the ring 8 threaded onto each rod 2, 3, forcing the ring 8 to be moved in the direction of the applied compressive force and thus locking the rod 2, 3 by the rollers 12′ constrained by the disk clamp 13′ against the inclined planes P1 of the internal trapezoidal chamber 14′ of each ring 8.

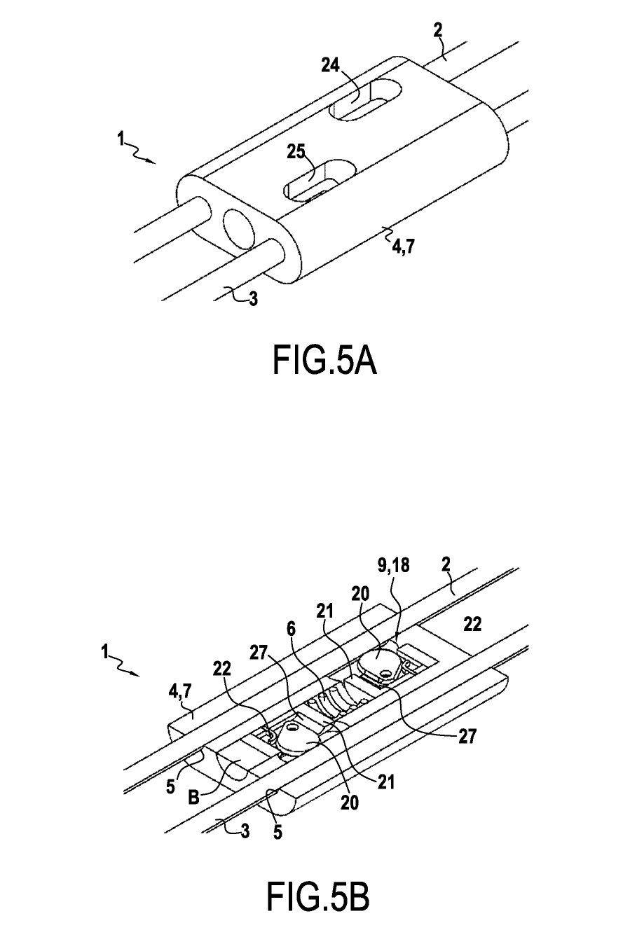

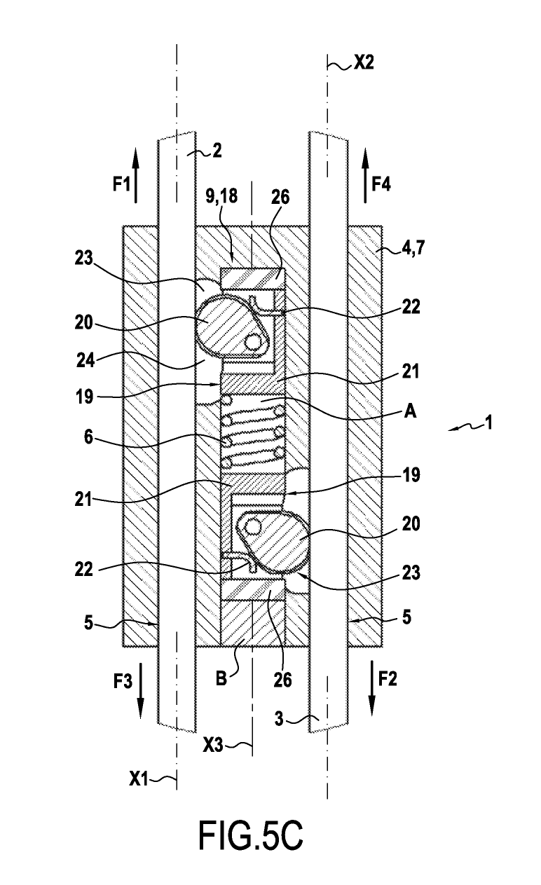

A last variant embodiment of the scoliosis correction device 1 of the invention is shown in FIGS. 5A to 5C.

In this variation, the distraction rods 2, 3 are introduced into the linking member 4 and extend along two parallel axes X1, X2, as in the variations of FIGS. 2A to 4B. The linking member 4 is again formed by a substantially parallelepipedal casing 7, comprising an upper access opening to the interior of the casing 7, this opening being closed by a closing plate screwed onto the body of the casing 7.

The device 1 comprises guide means in the first place comprising two channels 5 for inserting and guiding distraction rods 2, 3 cut parallel along the axes X1, X2 on either side of the casing 7 and a cushioning and locking structure comprising a device 18 for over-center locking of the distraction rods 2, 3 relative to the linking member 4.

The device 18 for locking the distraction rods 2, 3 acts as a general means 9 for locking rods and is inserted into the casing 7 in a cylindrical bore A pierced into the casing 7 between the channels 5 of the rods 2, 3 along a third axis X3 parallel to the axes X1, X2 of the distraction rods 2, 3 and coincident with the longitudinal medial axis of the casing 7. As can be seen in FIGS. 5B and 5C, the locking device 18 is held in a fastened position in its bore A by a plug B.

The locking device 18 advantageously comprises two keepers 19 that are movable in translation in the linking member 4 in a direction X3, said keepers 19 being disposed either side of a suspension element 6 in the bore A and each bearing firstly on the suspension member 6 interposed between them both and secondly on one of the distraction rods 2, 3, in order to lock it against sliding in a first direction by an over-center effect while being able to release them to slide in a second direction opposite to the first during stages of spine growth.

Thus, the keepers 19 of the locking device 18 are suspended on the suspension element 6, here a helical spring which, because of its intrinsic stiffness, exerts a pre-load on the keepers 19 that tends to urge them against the pads 26 at the ends of the bore A and thus to lock them in translation along the axis X3 in the bore A. Each of them comprises a cam 20 that pivots on a support body 21 bearing on the suspension element 6. In addition, the cam 20 of each keeper 19 cooperates with a resilient return element 22 attached to the support body 21 and constraining said cam to bear, via its cam surface 23, against one of the distraction rods 2, 3 in order to lock it against sliding relative to the linking member 4 in a direction opposite to the direction F1, F2 of elongation of the rods with growth of the inorganic patient's spine.

The cams 20 are pivotally mounted in a housing provided in their respective support body 21, which is cylindrical in shape, each extending to the outside of said support body 21 in a chamber 24 hollowed into the casing 7 and into which the cams 20 extend in contact with the outer surface of a distraction rod 2, 3 in order to rub on that outer surface via their cam surface 23 under the effect of leaf springs 22 that urge the cams in the direction of the rods 2, 3.

In order to encourage rubbing of the cam surface 23 on the distraction rods 2, 3, said cam surfaces 23 may be coated with a material with a high coefficient of friction, in particular an elastomeric material, for example.

As can be seen in FIG. 5A, during normal use of the correction device 1, the locking device 18 is covered by the closing plate of the casing 7. This closing plate may also optionally include ports 25, 26 providing access to the cams 20. These access ports 25 may in particular act to release the cams without having to open the casing 7, if required.

The function of the correction device 1 as described above and shown in FIGS. 5A to 5C is very simple and ultimately very similar to the function of the variations described above.

In fact, in the initial position and at rest, the distraction rods 2, 3 are each locked against sliding in the direction of the arrows F3, F4 relative to the casing 7 by the effect of the leaf springs 22 the stiffness of which is higher than the frictional force of the cams 20 of the keepers 19 of the locking device 18 on the rods 2, 3. The rods 2, 3 are thus locked by an over-center effect. In the event of a large force on the rods in the direction of the arrows F3 and/or F4, compression of the suspension element 6 nevertheless allows longitudinal movement of the keepers 19 inside the casing, providing the two distraction rods 2, 3 with longitudinal flexibility.

Consequently, during any growth stages of the spine equipped with the correction device 1, the spine then tends to exert a tensile force on each of the distraction rods 2, 3 in the direction of the arrows F1, F2. This tensile force induces a frictional force of the distraction rods 2, 3 on the cam surfaces 23 of the cams 20 that is greater than the stiffness of the leaf springs 22, which tend to urge the cams 20 back against the springs 22, which deform. Sliding of the rods 2, 3 in the direction of the arrows F1, F2 is then permitted. As soon as traction on the rods 2, 3 halts or becomes less than the stiffness of the springs 22, the rods 2, 3 are again locked in translation by the over-center effect of the cams 20 on these rods.

The correction device 1 of the present invention is thus entirely self-adapting in type and without any external surgical intervention once the device has been implanted in the patient.

Outside the stages of growth, the two distraction rods 2, 3 are locked against sliding under the effect of the locking means 9 employed in the variant embodiment under consideration from those described above and shown in FIGS. 2A to 5C. The correction device 1 then operates in compression and thus carries out its role of correcting the position of the spine. The suspension element 6 with which the correction device 1 is provided can thus accommodate compressive forces to the best possible extent, in order to provide the distraction rods, and thus the spine, with longitudinal flexibility, in accordance with anatomical reality. This longitudinal flexibility can essentially and advantageously be used to prevent vertebral arthrodesis phenomena in the scoliotic portions that have been instrumented for the purposes of correction.

The correction device 1 of the invention is principally but not essentially intended to be fitted to child patients between 3 and 15 years of age. In particular, a correction device as shown in FIGS. 1A to 1C is principally dedicated to adult patients or patients who have stopped growing, while a correction device 1 having the self-adapting devices as shown in FIGS. 2A to 5C is more particularly dedicated to instrumenting growing children.

In addition, with growing patients, the distraction rods 2, 3 used must necessarily be sufficiently long to constitute a reserve of rod necessary to accommodate the growth of the child for at least two years. In practice, since a child's spine grows by a maximum of 80 millimeters (mm), the correction device must have a reserve of rod of at least 50 mm, envisaging at most one change of rods (and fastenings), once the reserve has been exhausted, using new rods and fastenings that are suitable for the growing child.

Mechanically, the correction device 1 of the invention can accommodate a pre-load point force applied during fitting of 3000 newtons (N) in compression, as well as a distraction force of 1000 N in compression once implanted into the patient.

The correction device 1 of the invention must, of course, be capable of being sterilized easily and effectively and must be long-term biocompatible in a living organism. For this reason, its constituent elements are all constituted by a biocompatible material. In particular, the linking member 4 and the distraction rods 2, 3 are preferably constituted by a biocompatible metal such as titanium.

It must also have a minimal bulk so that is does not project from underneath the skin of the implanted patient, and it must be properly covered by the muscles. For this reason, the maximum length and width of the casing 7 for linking the distraction rods 2, 3 must be a maximum of 50 mm, with a thickness of at most 10 mm. The dimensions of the distraction rods 2, 3 themselves are those that are usual in the scoliosis treatment field, 3.5 mm to 5.5 mm in diameter.

Surgically, the technique for fitting the correction device of the invention does not in any way differ from the techniques for fitting more conventional equipment.

This is carried out under general anesthetic, under strict aseptic operating room conditions. The patient to be equipped with the device is positioned face down and incised posteriorly to the subcutaneous and musculoaponeurotic planes so that the vertebrae onto which the distraction rods 2, 3 of the correction device is to be connected can easily be seen. Using fastening screws and hooks, said rods are simply fastened onto the end vertebrae of the curvature of the spine that is to be corrected. Next, the length of said rods is adjusted in the linking member 4 of the correction device 1 so as to place the correction device in compression and to lock the distraction rods therein. To finish, the surgeon covers the correction device 1 with the muscle planes that have previously been moved to access the vertebrae in order to facilitate long-term tolerance of the equipment, then recloses the incision using conventional techniques.

1. A device (1) for correcting scoliosis, comprising at least:
a pair of distraction rods (2, 3); and
a distraction rod linking member (4) provided with means (8, 18) for guiding each of the rods relative to each other inside said linking member;
characterized in that it comprises, for each distraction rod:
at least one suspension member (6) for the distraction rods (2, 3) that is capable of providing flexibility and exerting a longitudinal pre-load on the distraction rods (2, 3); and
a structure for cushioning and locking the distraction rods in translation in the linking member, said structure being active only when compressive forces are applied by the spine of a patient to the distraction rods (2, 3) so as to absorb and oppose said compressive forces and augment the comfort of the patient, thereby preventing arthrodesis of the vertebrae of the patient's spine.

2. A scoliosis correction device according to claim 1, characterized in that the linking member (4) comprises a casing (7) inside which at least one said suspension member (6) for each rod is housed and the rods are inserted into said casing and each cooperates with a said suspension member.

3. A scoliosis correction device according to claim 1, characterized in that the distraction rods (2, 3) are introduced into and extend along two parallel axes (X1, X2) in the linking member (4).

4. A scoliosis correction device according to claim 3, characterized in that the structure for cushioning and locking the correction device comprises, for each distraction rod (2, 3), a ring (8) for adjusting the positions of the rods (2, 3) relative to the linking member (4), each said ring (8) being mounted coaxially and being slidably movable on each said rod, and cooperating with at least one means (9) for locking against sliding on the rods in at least one direction.

5. A scoliosis correction device according to claim 4, characterized in that each adjusting ring (8) bears on at least one said suspension member (6) mounted between an internal wall of the linking member (4) and said adjusting ring (8).

6. A scoliosis correction device according to claim 4, characterized in that the locking means (9) comprise a screw (10) inserted into an aperture formed in the adjusting ring.

7. A scoliosis correction device according to claim 4, characterized in that the locking means (9) comprise a toothed washer (11).

8. A scoliosis correction device according to claim 4, characterized in that the locking means (9) consist of an assembly comprising a split ring (12) and a disk clamp (13) slidably mounted on the distraction rod (2, 3) inside an at least partially conical chamber (14) formed in the adjusting ring (8), such that the disk clamp (13) presses on the split ring (12) in a conical section (15) of the chamber.

9. A scoliosis correction device according to claim 8, characterized in that the disk clamp (13) cooperates with a return means (16) housed in a non-conical section (17) of the chamber (14) formed in the adjusting ring (8).

10. A scoliosis correction device according to claim 4, characterized in that the locking means (9) consist of an assembly comprising rollers (12′) and a disk clamp (13′) slidably mounted on the distraction rod (2, 3) inside an at least partially conical chamber (14′) formed in the adjusting ring (8), such that the disk clamp (13′) presses on the rollers (12′) in a conical section of the chamber.

11. A scoliosis correction device according to claim 10, characterized in that the disk clamp (13′) cooperates with a return means (16′) positioned between the disk clamp (13′) and an internal wall of the chamber (14′) perpendicular to the axis (X1, X2) of the rod (2, 3).

12. A scoliosis correction device according to claim 9, characterized in that the return means of the disk clamp comprise a helical spring (16, 16′) mounted coaxially with the distraction rod (2, 3) in said chamber (14, 14′) of each ring (8).

13. A scoliosis correction device according to claim 1, characterized in that the cushioning and locking structure comprises an over-center device (18) for locking the distraction rods relative to the linking member.

14. A scoliosis correction device according to claim 10, characterized in that the device (18) for locking the distraction rods comprises two keepers (19) that are movable in translation in the linking member (4) in a direction (X3) parallel to the axes (X1, X2) in the linking member, said keepers being disposed either side of the suspension member (6) and each bearing both on said suspension member and also on a distraction rod (2, 3) in order to lock it against sliding in a first direction (F3, F4) relative to the linking member (4) by an over-center effect while being capable of allowing it to slide freely in a second direction (F1, F2) opposite to the first during stages of growth of a spine instrumented with the correction device.

15. A scoliosis correction device according to claim 11, characterized in that each keeper (19) of the locking device comprises a cam (20) pivoting on a support body (21) bearing on the suspension member (6), the cam cooperating with a resilient return element (22) that is attached to the support body (21) and that constrains said cam to bear on its cam surface (23) against a distraction rod.
